(19) United States
(12) Patent Application Publication
(10) Pub. No.: US 2024/0025925 A1
(43) Pub. Date: Jan. 25, 2024

(54) SYSTEMS AND METHODS FOR PREPARATION OF HIGHLY REACTIVE ALKALI METAL DENDRITES FOR THE SYNTHESIS OF ORGANOLITHIUM REAGENTS

(71) Applicant: THE TEXAS A&M UNIVERSITY SYSTEM, College Station, TX (US)

(72) Inventors: Andy A. THOMAS, College Station, TX (US); Michael P. CROCKETT, College Station, TX (US); Lupita S. AGUIRRE, College Station, TX (US); Leonel B. JIMENEZ, College Station, TX (US); Han-Hsiang HSU, College Station, TX (US)

(21) Appl. No.: 18/224,207

(22) Filed: Jul. 20, 2023

Related U.S. Application Data
(60) Provisional application No. 63/390,753, filed on Jul. 20, 2022.

(51) Int. Cl. C07F 1/02; C07C 29/70; C07F 5/02; C07C 1/32; C07C 45/44; C22C 1/00

(52) U.S. Cl. CPC C07F 1/02; C07C 29/70; C07F 5/027; C07C 1/328; C07C 45/44; C22C 1/00; B22F 9/16

(57) ABSTRACT

Systems and methods for formation of highly reactive alkali dendrites are provided. For example, in some embodiments alkali metals are dissolved in ammonia to form metal electrides after which the ammonia is removed via vacuum to reveal highly activated metal surfaces in the form of crystalline alkali dendrites. The alkali dendrites can mimic powders but have the advantage of being freshly prepared from inexpensive and readily available metal sources. These uniquely activated metals exhibit enhanced reactivity comparatively to similar off the shelf sources of the corresponding metals. For example, the dendrites can have about 100 times greater surface area than conventional metal sources and/or be about 19 times more reactive than powders that serve as the industry standard for the preparation of organometallic compounds. After surface activation, these metals can be used to prepare various organometallic reagents.

### CROSS REFERENCE TO RELATED APPLICATION

The present disclosure claims priority to and the benefit of U.S. Provisional Application No. 63/390,753, entitled “Preparation of Highly Reactive Lithium Metal Dendrites,” filed on Jul. 20, 2022, the content of which is incorporated by reference herein in its entirety.

### GOVERNMENT RIGHTS

This invention was made with government support under Grant No. DBI-0116835 awarded by The National Science Foundation. The government has certain rights in the invention.

### FIELD

The present disclosure relates to systems and methods for formation of highly reactive alkali dendrites, and more particularly relates to the surface activation of alkali metals with ammonia for synthesis of organometallic reagents.

### BACKGROUND

Lithium (Li) metal has revolutionized many scientific arenas from the development of active pharmaceuticals to Li-batteries. Specifically, the physical form of the Li-metal can greatly impact its chemical properties and function as most processes take place at the Li-metal surface. A person skilled in the art will recognize that Li-metal can be highly malleable and can be shaped into various workable forms such as pellets, rods, and foils. Although these physical forms of Li-metal find use in many real-life settings, the increased demand for faster electron transfer properties in several industrial applications has resulted in a great deal of investigation into Li-sources that vary in surface area and composition. For instance, one of the most significant advances in the chemical sciences involved the development of Li-powders for the preparation of organolithium compounds which continue to provide solutions to many pressing synthetic challenges to this day.

Commercial sources of lithium metal routinely provide avenues for the synthesis of many organometallic reagents. Technical aspects of such reactions can impede everyday synthetic operations on both small and large scale reactions, and have several shortcomings. For example, FIG. 1A illustrates a prior art process for using Li-metal to construct carbon-lithium bonds. In the realm of organometallic chemistry, the transfer of electrons from a metal to an organic substrate is the quintessential process to form carbon-metal bonds. However, because the reactions are heterogeneous, variabilities in the quality and area of the metal surface can often render these processes unpredictable and facetious in nature especially on routine laboratory scales. Historically, reaction development in this field has relied on activating the metal by mechanically reducing the size of the metal particle and by the addition of chemical activators such as iodine to clean the metal surface.

Currently, commercial access to Li-powders or dispersions has become extremely limited as lithium's broad impact and future potential in the energy and battery sector has shifted production lines of raw materials to other areas.

Moreover, processes for the preparation of lithium powder (dispersion), which is the widely used source of lithium for application, are obtained by physical means, while heating the alkali metal at a temperature above its melting point, and dispersing the molten metal in mineral oil, making the overall process unsafe and impracticable in most academic settings. For example, in the 1970s, Rieke pioneered an elegant solution to these practical problems of efficiency and generality for several alkaline earth and transition metals such as magnesium, zinc, and copper by developing a method that allowed access to highly reactive metal powders via the reduction of metal salts with alkali metals, as shown in FIG. 1B. One shortcoming of lithium, however, is that Li-metal bears the lowest reduction potential among the elements and therefore cannot be prepared using Rieke's method.

Accordingly, there is a need for development of systems and methods for formation a highly reactive lithium-metal source that mimics lithium powders but has the advantage of being freshly prepared from inexpensive and readily available lithium sources.

### SUMMARY

The present application is directed to systems and methods for formation of highly reactive alkali dendrites. Formation of these dendrites can result from dissolving alkali metals in ammonia. For example, in some embodiments, a lithium-rod can be dissolved in liquid ammonia to form metal electrides. Once formed, the ammonia can be removed to synthesize a new lithium-metal source. In some embodiments, the new lithium-metal source can be in the form of crystalline lithium-dendrites having highly activated metal surfaces. These uniquely activated metals can exhibit enhanced reactivity comparatively to similar off the shelf sources of the corresponding metals. For example, these lithium-dendrites can have about 100 times greater surface area than conventional lithium-sources created by prototypical mechanical activation methods. Concomitant with the surface area increase, the lithium-dendrites are 19 times more reactive than lithium-powders which are currently the industry standard for the preparation of organolithium compounds. After surface activation, these metals can be used for efficient synthesis of various organometallic reagents, can serve as reducing agents, or any chemistry traditionally associated with alkali earth metals. In some embodiments, the presently disclosed methods can allow for preparation of various families of organometallic reagents utilizing the described metals.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

This disclosure will be more fully understood from the following detailed description, taken in conjunction with the accompanying drawings, in which:

FIG. 1A is a prior art illustration of a reaction for generating metal-carbon bonds;

FIG. 1B is a prior art illustration of a reaction for activation of metal for metal-carbon bond formation;

FIG. 2A is an example reaction of the present embodiments for forming lithium (Li)-dendrites from Li-ammonia solutions;

FIG. 2B is a photograph of a Li-rod used in the reaction of FIG. 2A;

FIG. 2C is a photograph of an electride formed as a result of the reaction of FIG. 2A;

FIG. 2D is a photograph of the Li-dendrites formed from the electride of FIG. 2C via the reaction of FIG. 2A;

FIG. 2E is a photograph of the Li-dendrites of FIG. 2D in a flask;

FIG. 2F is another example reaction of the present embodiments for forming alkali dendrites;

FIG. 3A is a photograph that compares the surface areas of 500 milligrams of each of the Li-rod, Li-foil, Li-powder, and the Li dendrites;

FIG. 3B is a scanning electron microscopy (SEM) image of Li-dendrites of FIG. 2D at 60× magnification;

FIG. 3C is a SEM image of Li-dendrites of FIG. 2D at 250× magnification;

FIG. 3D is a SEM image of Li-dendrites of FIG. 2D at 500× magnification;

FIG. 3E is a SEM image of Li-powder at 1100× magnification;

FIG. 4A is a reaction and table showing kinetic measurements carried out by rapidly injecting a hexane solution of isopropanol (3.0 mmol) into Li-metal (2.0 mmol) suspensions in hexanes;

FIG. 4B is a reaction and table evaluating experiments conducted over a range of reaction scales and their corresponding yield percentages;

FIG. 4C is a graph illustrating the reactivity of the compounds of FIG. 3A following the reaction of FIG. 4A;

FIG. 5 is a table illustrating preparation of various organolithium reagents and their use in further transformations.

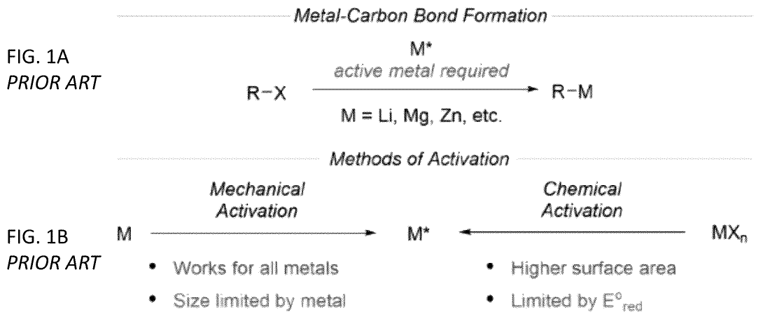

### DETAILED DESCRIPTION

Certain exemplary embodiments will now be described to provide an overall understanding of the principles of the structure, function, manufacture, and use of the devices and methods disclosed herein. This includes in the description and claims provided for herein. Further, one or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

The present disclosure generally relates to systems and methods for the preparation of highly reactive alkali dendrites with highly activated surfaces from alkali metals. At least one novel aspect of the present disclosure provides systems and methods for the activation of Li-metal that furnishes a crystalline material with highly increased reactivity. One exemplary method of the present embodiments can use liquid ammonia to dissolve Li-metal to furnish a new Li-metal source in the form of crystalline Li-dendrites. The processes by which the Li-metal reacts to form the Li-dendrites are discussed in detail below. The Li-dendrites can have approximately 100 times greater surface area than conventional Li-sources created by prototypical mechanical activation methods. Moreover, these Li-dendrites can be highly reactive and mimic Li-powders while having the advantage of being freshly prepared from inexpensive and readily available Li-sources. After surface activation, these metals can be used to synthesize organometallic reagents. These families of organometallic reagents can, in some embodiments, be used for synthetic transformations of various compounds.

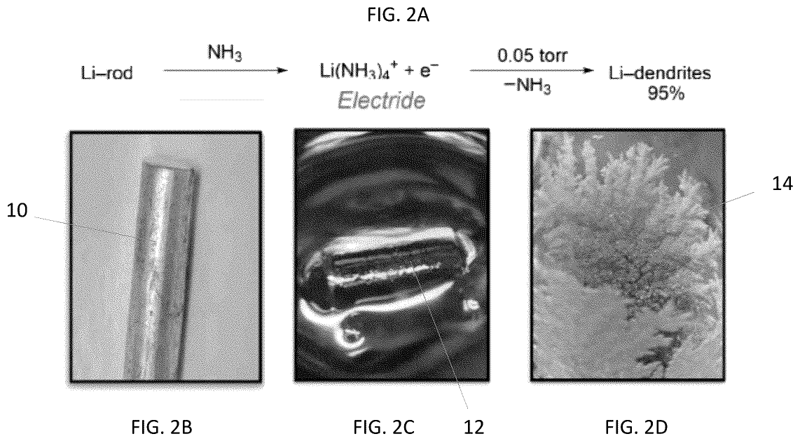

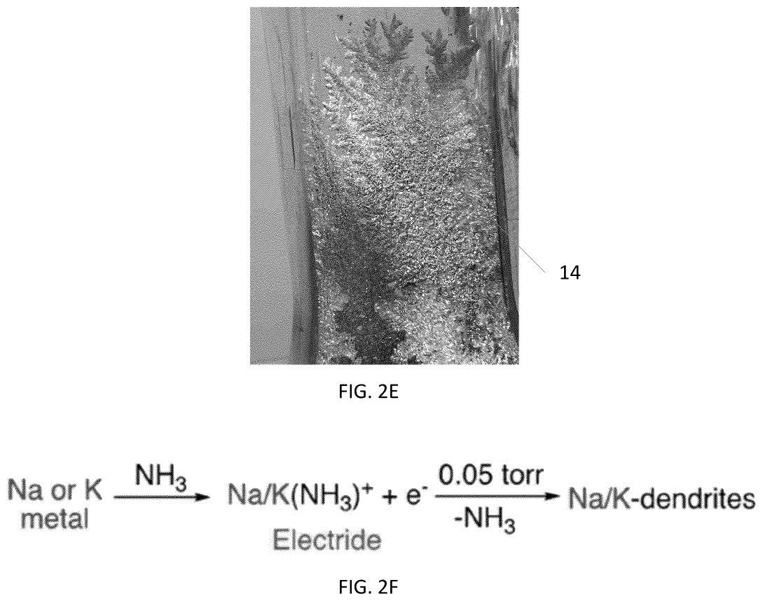

FIGS. 2A-2D illustrate an example reaction for the formation of Li-dendrites that can be plated and/or crystallized from Li-ammonia solutions in greater detail. As shown in FIG. 2A, synthesis of Li-dendrites from bulk Li-material, e.g., Li-metal, and ammonia can involve a two-step process. First, the low reduction potential of lithium can be leveraged by creating a chemical equilibrium which would allow for Li-metal to be briefly ionized to a Li-cation and electride. FIGS. 2B-2C illustrate example embodiments of the Li-metal and the electride, respectively. As shown, the Li-metal can include a Li-rod 10, though in some embodiments, a Li-foil can be used. The low reduction potential of Li can allow for it to be easily ionized in the presence of strongly Lewis basic ligands such as ammonia or alkylamines. For example, the Li-rod 10 can be condensed in liquid ammonia to form the lithium electride. During this type of dissolution process, the s electron from Li-metal can be ionized into solution which can lead to the characteristic blue hue of a solvated electron under relatively dilute conditions and a fiery bronze color at high concentrations. An example of the alloy, e.g., the Li-bronze compound or solution 12, is shown in FIG. 2C.

The ammonia used in the reaction of FIG. 2A can be manufactured annually on megaton scales making it an inexpensive and ideal solvent for both academic and industrial settings. Although it is a gas at standard atmospheric temperatures and pressures, its enthalpy of vaporization (23.35 KJ/mol at room temperature (rt)) is high, making it simple to condense and easy to handle in liquid form. In some embodiments, 0.5 g Li-metal/13 mL was found to dissolve at −78 ° C. in anhydrous ammonia to create a Li-bronze solution. The temperature of −78 ° C. can be kept and maintained substantially constant throughout the addition of liquid ammonia to the metal alloy followed by slow stirring of the bronze solution. The solution can then be allowed to reach room temperature (after all the ammonia is added) while being stirred at a constant rate to evaporate small amounts of ammonia.

Once formed, the electride can be followed by the microscopic reverse to reform the metal in a more reactive state. That is, after warming to room temperature along with vigorous stirring, the ammonia can slowly be removed in vacuo at 0.05 ton to remove substantially all of the liquid ammonia present in the alloy to reveal highly activated metal surfaces. For example, in some embodiments, an amount of the ammonia removed from the alloy can be in approximately a range of about 99% to about 100% with respect to a total amount of the ammonia in the alloy, or in approximately a range of about 99.5% to about 100% with respect to a total amount of the ammonia in the alloy. It will be appreciated that in some embodiments trace amounts of lithium amide may be left behind as known to one skilled in the art. For example, in some embodiments, when the ammonia is removed, a metallic Li-mirror can be gradually deposited on the walls of the vessel. Upon further evaporation, e.g., about 10-15 min, of ammonia, metallic Li-dendrites 14 can begin to grow, forming the semicrystalline material, as shown in FIG. 2D. After the removal of exogenous ammonia via vacuum for about 0.5 h, the Li-dendrites 14 can be used immediately or easily removed, collected, weighed, and/or stored under inert atmospheres, e.g., inert argon atmospheric conditions, without significant damage to the surface or morphology of the material. It will be appreciated that it may be important to avoid contamination of the lithium sources to oxygen as it will reduce the reactivity of the dendrites. Moreover, this process can be reproducible and scalable up to at least 200 mmol where similar Li-dendrites were observed, as discussed in greater detail below. The ability to prepare a highly activated metal surface at the beginning of each reaction can provide the opportunity to have a better yield and provides a controlled process where conventional processes fail. As shown in FIG. 2E, the Li-dendrites 14 can resemble Li-mirrors or crystals that are formed on a flask in which they are contained upon the removal of ammonia from Li-electride solutions.

A novelty of the instantly disclosed systems and methods for the preparation of highly reactive alkali dendrites relies on reversibility of electride formation. Formation of electride solutions being a reversible process may be unexpected as these solutions can be unstable. Moreover, the reactions that use alkali metal dendrites produce desired results in the absence of nearly all protic compounds to yield the desired products in target amounts. Because ammonia is a protic compound, it can be an unexpected result to one skilled in the art that highly reactive alkali dendrites may be prepared from ammonia as the presence of ammonia may conventionally be thought to inhibit the desired reaction.

FIG. 2F illustrates an alternate example reaction for the formation of Li-dendrites from Li-ammonia solutions. For example, while the methods of the present embodiments are discussed with respect to formation of Li-dendrites, in some embodiments, this general reaction can be applied to other alkali metals, such as sodium and potassium. As shown in FIG. 2F, the reaction can include a sodium or potassium metal being dissolved in liquid ammonia in lieu of lithium. This dissolution can create an electride that forms a Na-dendrite or a K-dendrite in accordance with the reaction discussed above with respect to lithium.

Solvents other than ammonia can also be used in the reaction of the present embodiments. Some non-limiting examples of such solvents can include Hexmethylphosphoramide (HMPA) and/or different degrees of amines, among others. These solvents can be used with Li-metal, Na-metal, and/or K-metal, among others.

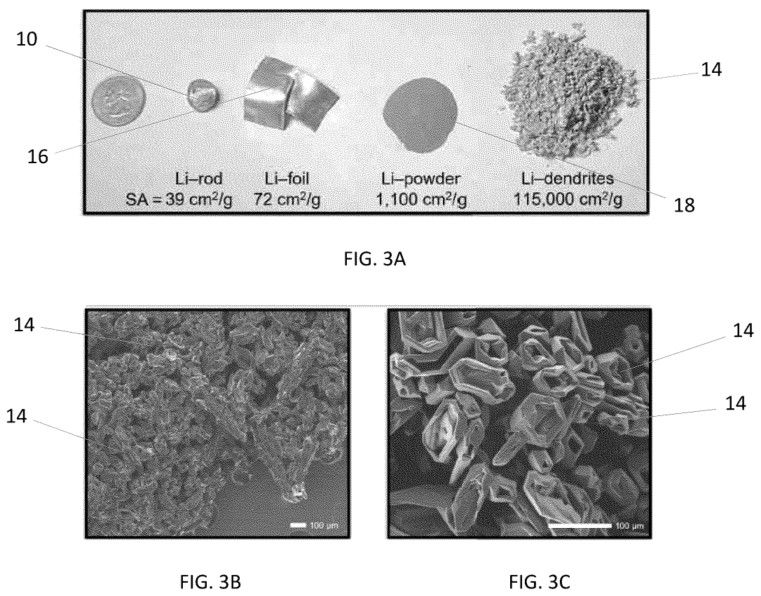

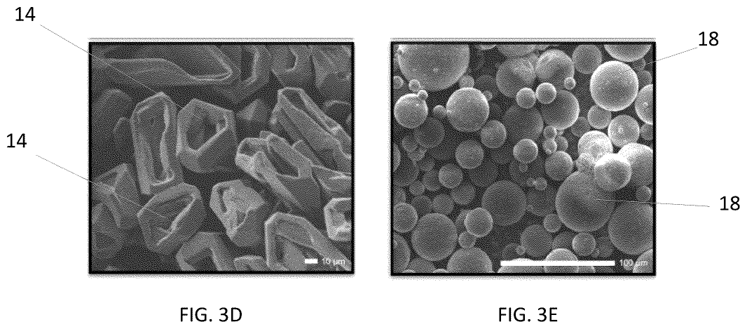

The ability to dissolve alkali metals provides the unique ability to create a highly activated and clean surface of the alkali dendrite after the removal of ammonia. Specifically, the metallic state of the Li-dendrites of the present embodiments after formation can exhibit a clean surface as compared to that of other Li-metals. Moreover, the surface area of the Li-dendrites 14 of the present embodiments can be significantly greater than the surface areas of the off-the-shelf Li-rod 10, Li-foil 16, and/or Li-powder 18 discussed herein. FIG. 3A illustrates a comparison of the surface areas of Li-rod 10, Li-foil 16, Li-powder 18, and Li-dendrites 14 with a quarter used as a point of reference. As shown, Brunauer-Emmett-Teller (BET) surface area measurements of the Li-dendrites (115,000 cm²/g) revealed that the material has, on average, about 100 times higher surface area than the Li-powder (1,100 cm²/g), and in some embodiments about 105 times higher surface area than the Li-powder. This increased surface area can allow the Li-dendrites 14 of the present embodiments to be used in a variety of applications, such as lithium-ion batteries, preparation of organolithium compounds, and other technical advances that utilize lithium. To gain insight into the surface morphology of the Li-dendrites 14, a sample for scanning electron microscopy (SEM) can be randomly selected and carefully mounted under inert atmospheres.

FIGS. 3B-3E illustrate comparison images of the Li-metals at various magnifications. Although the bulk morphology can be agglomerated in a dendritic form, for example, the SEM images of the Li-dendrite across magnifications, as shown in FIGS. 3B-3D, can show that the microstructure can include non-porous crystals in the range of about 20-100 μm in diameter. Moreover, the crystals can exhibit significant amounts of screw and step dislocations which can be attributed to nucleation being disturbed during the ammonia removal process. Further still, a comparison of the microstructure of the Li-dendrites to Li-powder, with the Li-powder shown in FIG. 3E, showed that the morphology of the Li-powder can be spherical with no significant crystalline features.

### EXPERIMENTS

### Kinetic Analysis of Li-Metal in Various Physical Forms

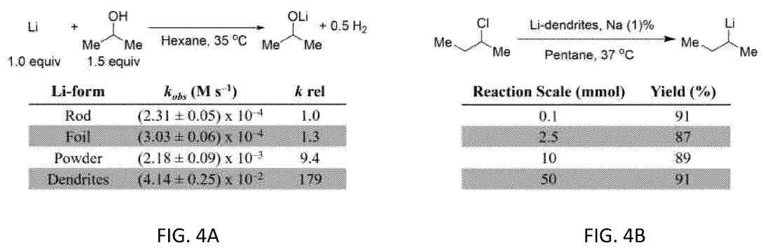

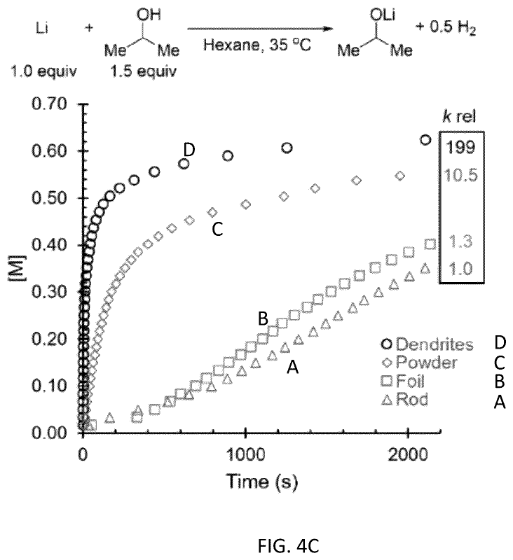

A comparison of reactivity of synthesized Li-dendrites 14 to Li-metal in other physical forms can be performed via kinetic analysis. FIGS. 4A-4C illustrate the results of this comparison in greater detail. To establish the kinetic behavior, isopropanol can be added separately to hexane suspensions of lithium rod, foil, powder, and dendrites, as shown in the reactions of FIGS. 4A-4B, or injected as a hexane solution of isopropanol (3.0 mmol) into Li-metal (2.0 mmol) suspensions in hexanes, such that the evolution of hydrogen gas can be visually monitored by video recording using a burette. It will be appreciated that the experiments with these suspensions can be conducted over a range of reaction scales under identical conditions to generate the results tabulated in FIGS. 4A-4B. This analysis can reveal sigmoidal concentration versus time curves for the rod, foil and dendrites, as shown in FIG. 4C. To allow for a straightforward comparison, the rate of hydrogen formation can be measured through the linear region of each curve. In some embodiments, the rate of hydrogen formation can be first measured with curve (A) of the Li-rod (2.08 ±0.05)×10⁻⁴ M/s (A) so that the rates can be normalized. While lithium rod 10 and lithium foil 16 both resulted in near identical reaction rates in curve (A) and curve (B), respectively, Li-powder 18 in curve (C) was about 10.5 times faster than the Li-rod 10. Unexpectedly, the Li-dendrites 14 (curve (D)) can exhibit a reactivity of about 199 times more reactive than the rod (curve (A)), and about 19 times faster than the Li-powder 18 (curve (C)), as shown in FIG. 4C. These higher reactivity values can demonstrate that the Li-dendrites 14 can provide a superior reaction surface over the other Li-metal sources. Moreover, not only was the overall reaction faster with the Li-dendrites 4, but the induction period can be significantly reduced over the induction period of either of the Li-rod 10 and foil 16. This increase in reactivity rate can be due to the combination of clean and increased surface areas as well as the high densities of dislocations and imperfections which are can accelerate rates of electron transfer.

### Synthesis of Reagents with Li-Dendrites

In some embodiments, the Li-dendrites can be highly reactive toward oxidative addition reactions with organic halides allowing access to a wide variety of organolithium species that previously could only be prepared with Li-powders. The protocol to activate the Li-metal can be performed over a range of reaction scales allowing for fresh batches of activated Li-metal to be prepared. The preparation of alkali metals or alloys, e.g., Li-dendrites 14, with highly activated surfaces can provide a technical advance by affording a metal source that allows for various efficient synthesis of organometallic reagents on varying scales. Organolithium reagents synthesized with the Li-dendrites of the present embodiments can exhibit superior yields than conventional sources of lithium metal. For example, in some embodiments, yields can be improved by about 5% to about 25% as compared to organolithium reagents synthetized with conventional sources of lithium metal. It will be appreciated that while the present disclosure discusses organolithium reagents, other organo-alkali metal reagents, e.g., organosodium and/or organopotassium, can be synthesized with the techniques of the present disclosure.

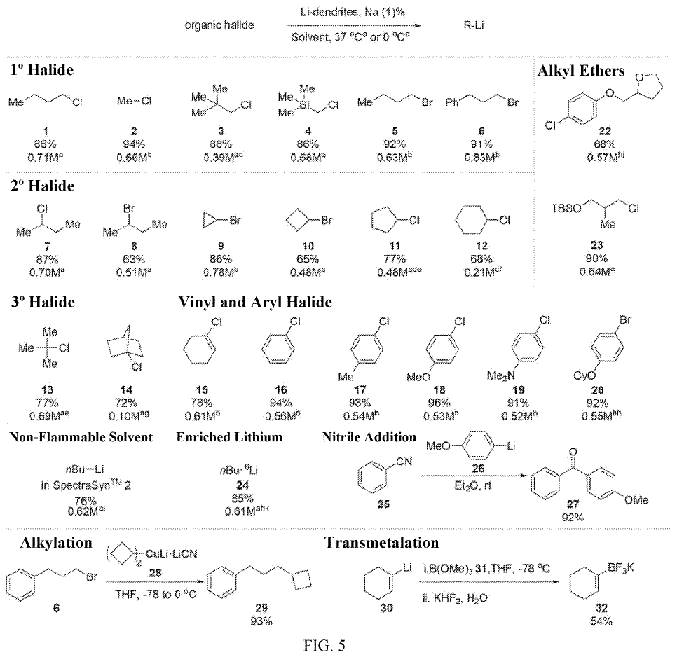

FIG. 5 illustrates preparations of organolithium reagents and use in further transformations via use of various compounds, e.g., organic halides, in greater detail. For example, the Li-dendrites 14 of the present embodiments can leverage their increased reactivity to synthesize organometallic reagents on various scales, e.g., small, medium, and even large scales. For example, the volume of the Li-dendrites can allow for minute amounts of Li-metal to be reliably and accurately weighed affording reproducible results even on small scales, which is often challenging for Li-insertion reactions. Moreover, the activated surfaces allow for the preparation of many organometallic reagents that are traditionally challenging to prepare by any other means, such as lithiated ethers 22 and 23.

As noted above, in some embodiments the Li-dendrites can be highly reactive toward reductive insertion reactions with organic halides allowing access to a wide variety of organolithium species that previously could only be prepared with Li-powders. Moreover, the enhanced reactivities of the Li-dendrites 14 can be used to develop a simple protocol for the preparation of organolithium reagents. To test the feasibility of the outlined reductive metalation, s-BuCl can be selected as the model substrate because its rate of Li-metal insertion is known to be in between that of n-BuCl and t-BuCl making it ideal for reaction optimization. After an extensive survey of the reaction conditions, s-BuLi was found to be able to be produced in 87% yield employing 4.0 equivalent of Li-dendrites along with 1 mol% of sodium on a 2.5 mmol scale. The addition of sodium (−1 mol%) as well as the employment of multiple equivalents of Li-metal can be vital for reasonable rates of Li-insertion to be achieved and prevent deleterious elimination and or dimerization pathways.

Effect of Li-Metal Source on the Rate of Metalation with s-BuCl. Given the rate enhancement observed toward hydrogen evolution, a similar rate enhancement may be observable with reductive metal insertion reactions. For example, to establish the kinetic relationship between Li-powders 18 and Li-dendrites 14, s-BuCl solutions in pentane can be separately added to suspensions containing either the Li-dendrites 14 or Li-powder 18. Subsequent tracking of the s-BuCl concentration over time can allow for a direct comparison of the two materials. During such tracking, the Li-dendrites 14 can be found to have initiated and completely consumed the halide starting material in less time (about 300 s) than the Li-powder 18 usually can take to initiate. Further, upon fitting the linear region bearing constant k_{obs} the Li-dendrites 14 can insert at least about 6.2 times faster than the Li-powder 18. These results together can indicate that the Li-dendrite 14 would be ideally suited for applications that require faster or more predictable initiation rates.

A person skilled in the art will recognize that evaluation of the reliability and scalability of the optimized protocol can be performed over a range of reaction scales that are common practice in the synthetic laboratory, e.g., 0.1, 2.5, 10 and 50 mmol scales. As shown in FIG. 5, compounds such as n-BuLi could be readily prepared in comparable yields in a safer non-flammable poly(a-olefin) SpectraSyn™2, further highlighting the robustness of the formation of the Li-dendrites of the present embodiments. Moreover, with reference to FIG. 5, methyl chloride 2, neopentyl chloride 3, and trimethylsilylmethyl chloride 4 can result in high yields (86-94%). Although primary alkyl bromides 5 and 6 can be found to give the desired insertion reaction in excellent yields (>91%), the secondary alkyl bromide substrate 8 can result in slightly lower yield (63%) emphasizing that secondary bromide leaving groups offer avenues for nonproductive E2-elimination pathways to become competitive. Next, for a set of halocarbocycles (9-12), the reaction can proceed smoothly with the corresponding Li-carbocycle being formed in moderate to good yields (65-86%). A slight modification may be needed for the 5- and 6-membered halocarbocycles whereby tert-butyl methyl ether (TBME) can be identified as a sacrificial additive to prevent unproductive consumption of the organic halide. This method can be further applied to the synthesis of tertiary alkyl lithium reagents, which can be difficult to access using previously reported Li-insertion reactions on laboratory scales. Like the 5- and 6-membered ring systems, t-BuCl 13 can benefit greatly from the addition of TBME (77% vs. 45% without). Norbornyl chloride 14, on the other hand, can display moderate yields (72%) and show no improvement with TBME, consistent with TBME's role in the suppression of E2-elimination.

To broaden the utility of the Li-dendrite system, a series of vinyl and aryl chlorides can also be used. For example, after a solvent change to diethyl ether, 1-chlorocyclohexene 15 can result in good yields of the vinyl lithium reagent. Further, chlorobenzene 16, 4-chlorotoluene 17, 4-chloroanisole 18, and 4-chlorodimethylaminobenzene 19 can result in excellent yields (91-96%). Moreover, aryl bromide 20, a substrate known to undergo facile side reactions from lithium halogen exchange, can be found to afford its corresponding aryl lithium in excellent yields (92%). By investigating the aryl halide substrate scope, some limitations, such as strongly electron deficient arenes, e.g., 4-chlorobenzotrifluoride 21, can be observed to be completely unreactive under the presently described conditions.

To further establish the versatility of the methods of the present disclosure, preparation of organolithium compounds can bear functional groups that are often incompatible with alkyl lithium reagents such as t-BuLi, which may be the only viable option for complementary Li-halogen exchange processes. Notably, ethers can be often susceptible to facile deprotonation reactions with strong organolithium bases, and this limits their applications in synthesis. Accordingly, chloroarene 22 that bears both alkylether and tetrahydrofuran functional groups can undergo Li-insertion cleanly and efficiently. Moreover, primary alkyl chloride 23 incorporating an alkylether subunit can undergo smooth conversion into its organolithium, highlighting the robustness of our Li-source to create disconnections that have been employed in the synthesis of biologically relevant compounds.

Preparation of isotopically labeled n-Bu⁶ Li with Li-dendrites. It will be appreciated that the ability to achieve high yields from Li-insertion reactions on small scales with short reaction times can be of great synthetic importance. The methods of the present embodiments can be useful to synthesize ⁶ Li isotopically labeled organolithium reagents which can be used to obtain detailed aggregate structural information by ⁶ Li NMR investigations. For example, the high yields and concentrations obtained for n-Bu⁶ Li 24, a ubiquitous base can be used to prepare other organolithium congeners through Li-halogen exchange reactions, as shown in FIG. 5. Several contemporary protocols for the synthesis of 24 can use either the reduction of toxic diorganomercury compounds with ⁶ Li-rod or costly syntheses on scales of >100 mmol for high yields and purities to be achieved, which can exhibit several significant shortcomings over the instantly recited process.

In some embodiments, once the organolithium reagents discussed above are prepared, these reagents can be used in one or more synthetic settings. For example, the practical and synthetic advantages of Li-dendrites 14 over other Li-sources in the synthesis of various organolithium compounds can be used in synthetic transformations as shown in FIG. 5. In some embodiments, prototypical carbon-carbon bond forming processes such as nitrile addition can perform well. Further, the alkylation of 4-phenyl-1-bromobutane 6 can be simple, clean, and high yielding, e.g., a yield of about 90% or more, with about 93% in some embodiments, while employing the freshly prepared cyano-Gilman reagent 28. Finally, potassium trifluoroborate 32, which is a common reagent employed in Suzuki-Miyaura cross-coupling reactions, can also be isolated in good yields through trapping vinyllitihum 30 with trimethyl borate 31.

### PROCEDURES

### Procedure for Measuring the Solubility of Lithium in Ammonia

In an argon atmosphere glovebox, a 250 mL Schlenk flask was charged with lithium metal rod (500.0 mg, 72.0 mmol), a glass-coated stir bar, and capped with a septum. The flask was removed from the glovebox and placed under argon atmosphere on a Schlenk line. The Schlenk flask was placed in a dry ice bath (−78 ° C.) at a low stirring rate. A modified-graduated cylinder was placed under an argon atmosphere and submerged in a dry ice bath (−78 ° C.) until 13.0 mL of liquid ammonia was condensed. Using a cannula transfer, liquid ammonia was added to the Schlenk flask containing the lithium metal. The solution can be stirred in a dry ice bath until all the lithium metal rod was completely dissolved and a dark purple/blue color was achieved.

### General Procedure for the Preparation of Lithium Dendrites

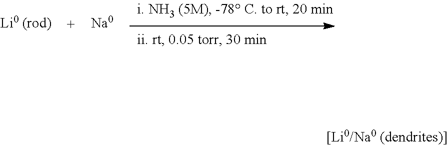

In the glovebox, a 50 mL Schlenk flask was equipped with a glass-coated stir bar, lithium (276 mg, 40.0 mmol), sodium (2.8 mg, 0.12 mmol, 1 wt %) and capped with a septum. The flask was removed from the glovebox and freshly condensed NH₃ (8.0 mL, 5 M) was added at −78 ° C. via cannula. After about 5 min, the lithium bronze solution was warmed to room temperature over 20 min where ammonia in an amount of about 2.0 mL boiled off slowly. The vessel was placed under vacuum (0.05 torr) to remove the remainder of ammonia over 30 min. The flask was backfilled with argon and returned to the glovebox. The lithium was removed by carefully scraping the walls of the flask and pouring onto wax weighing paper to furnish the lithium dendrites (265 mg, 95%) as a crystalline solid. The lithium dendrites kept their luster for several weeks in a sealed container in the glovebox though most were used within 24 h.

### Procedure for Measuring the Rate of Hydrogen Evolution with Various Lithium Sources

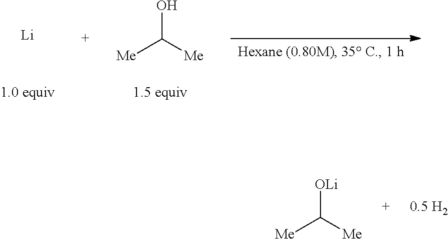

In the glovebox, a 10 mL Schlenk flask was equipped with a glass-coated stir bar, lithium (13.9 mg, 2.0 mmol, 1.0 equiv) and capped with a septum. The flask was removed from the glovebox and hexane (2.0 mL) was added via syringe. The flask was lowered into a 35 ° C. bath and the flask was quickly vented to release excess pressure. The flask was connected to a burette with a water reservoir (colored with CuSO₄) and stirring (400 rpm) was started. Video recording was started, the flask was opened through the sidearm, and a solution of isopropanol (0.50 mL, 3.0 mmol, 1.5 equiv, 6.0 M) in hexane was added rapidly via syringe. Recording of the reaction was continued until at least 14.0 mL of gas had been collected or 1 h had elapsed. Time points for every 0.50 mL were then extracted by going frame by frame in the resulting videos and subtracting the time from the end of the addition of IPA. The amount of hydrogen evolved at each timepoint was used to calculate an effective concentration of LiOiPr which was then plotted vs. time. The linear region, following any induction period, was fit using the Curve Fitter Toolbox in Matlab. This procedure was performed in triplicate to obtain an average rate for each form of lithium, with results shown and discussed with respect to FIG. 4C above. Compiled kinetic data for the formation of LiOiPr using lithium rod, foil, powder and dendrites is shown in Table 1, below:

| Entry | Run | k_{obs} (M s⁻¹) | k_{avg} (M s⁻¹) | k rel |
|---|---|---|---|---|
| Rod | 1 | (1.92 ± 0.01) × 10⁻⁴ | (2.08 ± 0.05) × 10⁻⁴ | 1.0 |
| | 2 | (1.98 ± 0.07) × 10⁻⁴ | | |
| | 3 | (2.33 ± 0.06) × 10⁻⁴ | | |
| Foil | 1 | (2.46 ± 0.04) × 10⁻⁴ | (2.71 ± 0.06) × 10⁻⁴ | 1.3 |
| | 2 | (2.46 ± 0.06) × 10⁻⁴ | | |
| | 3 | (3.21 ± 0.07) × 10⁻⁴ | | |
| Powder | 1 | (1.94 ± 0.06) × 10⁻³ | (2.18 ± 0.09) × 10⁻³ | 10.5 |
| | 2 | (2.37 ± 0.05) × 10⁻³ | | |
| | 3 | (2.22 ± 0.13) × 10⁻³ | | |
| Dendrites | 1 | (4.18 ± 0.30) × 10⁻² | (4.14 ± 0.25) × 10⁻² | 199 |
| | 2 | (3.54 ± 0.22) × 10⁻² | | |
| | 3 | (4.69 ± 0.22) × 10⁻² | | |

### Procedure for Measuring the Rate of s-BuCl Consumption with Various Lithium

Sources

In the glovebox, a 10 mL Schlenk flask was equipped with a glass-coated stir bar, lithium (69.4 mg, 10.0 mmol, 4.0 equiv) and capped with a septum. The flask was removed from the glovebox and pentane (2.5 mL) was added via syringe. The flask was lowered into a 30° C. bath. After about 5 min, a 1.0 mL stock solution containing s-BuCl (2.5 mmol, 1.0 equiv, 2.5M) and octadecane (6.25 mmol, 0.25 equiv, 0.625 M) in pentane was added rapidly. Aliquots (30-50 μL) were withdrawn at the indicated timepoints and quenched by rapidly injecting them into test tubes containing precooled (0° C.) water (3.0 mL). Each aliquot was extracted with pentane (2.0 mL), filtered through sodium sulfate, and analyzed by GC-FID. Relative integrations to the octadecane standard were used to find the s-BuCl concentration which was then plotted vs. time. The linear region, following any induction period, was fit using the Curve Fitter Toolbox in Matlab. This procedure was performed in duplicate to obtain an average rate for both forms of lithium.

### Procedures for the Preparation of Sec-Butyl Lithium on Alternate Scales

0.1 mmol scale: In the glovebox, a 1-dram vail was equipped with a stir bar, charged with lithium dendrites (2.8 mg, 0.40 mmol, 4.0 equiv), and capped with a septum cap. The vial was removed from the glovebox and placed in a preheated 37° C. bath. Pentane (0.40 mL) was added via syringe and stirring was started. A solution of sec-butyl chloride (100 μL, 0.1 mmol, 1 equiv, 1 M) in pentane was added dropwise over 5 min. The solution was stirred for 1 h. The solution was withdrawn via syringe and the vial was rinsed with pentane (2×100 μL). The combined pentane solution was added to an NMR tube containing cyclooctadiene (8.3 mg, 0.077 mmol). The yield was determined by a previously reported NMR method. Integration of the alkyllithium reagent (−0.98 ppm, 1H) relative to the cyclooctadiene alkene signal (5.47 ppm, 4H) resulted in a ratio of integrals: 2.95:10 for a yield of 91%.

2.5 mmol scale: In the glovebox, a 10 mL Schlenk flask was equipped with a glass-coated stir bar, lithium (69.4 mg, 10.0 mmol, 4.0 equiv), sodium (0.7 mg, 0.03 mmol, 1 wt %) and capped with a septum. The flask was removed from the glovebox and freshly condensed NH₃ (2.0 mL, 5 M) was added at −78° C. via cannula. After about 5 min, the lithium bronze solution was warmed to room temperature over 20 min where ammonia in an amount of about 0.5 mL boiled off slowly. The vessel was placed under vacuum (0.05 torr) to remove the remainder of ammonia over 30 min providing lithium dendrites. The flask was backfilled with argon and the septum was removed briefly about 10 s followed by carefully scraping the lithium dendrites off the walls to the bottom of the vessel with a metal spatula. The flask was lowered into a preheated oil bath at 37° C. Pentane was added (1.5 mL, 1 M) followed by the dropwise addition of a sec-butyl chloride solution (1.0 mL, 2.5 mmol, 1.0 equiv, 2.5 M) over 60 min with vigorous stirring. After a minimum of about 1 h after the addition, the purple/black heterogeneous mixture was then withdrawn with a syringe and the remaining solids were washed with pentane (2×1.0 mL). The combined pentane solution was filtered through a Teflon syringe filter (Restek Cat#26142-248, 13 mm, 0.22 μm). The resulting solution was titrated following the modified Gilman's method described above to provide 3.02 mL of a 0.70 M solution for an 87% yield.

10 mmol scale: In the glovebox, a 50 mL Schlenk flask was equipped with a glass-coated stir bar, lithium (276 mg, 40.0 mmol, 4.0 equiv), sodium (2.8 mg, 0.12 mmol, 1 wt %) and capped with a septum. The flask was removed from the glovebox and freshly condensed NH₃ (8.0 mL, 5 M) was added at −78° C. via cannula. After about 5 min, the lithium bronze solution was warmed to room temperature over 20 min where ammonia about 2 mL boiled off slowly. The vessel was placed under vacuum (0.05 torr) to remove the remainder of ammonia over 30 min providing lithium dendrites. The flask was backfilled with argon and the septum was removed briefly after about 10 s followed by carefully scraping the lithium dendrites off the walls to the bottom of the vessel with a metal spatula. The flask was lowered into a preheated oil bath at 37° C. Pentane was added (6.0 mL) followed by the dropwise addition of a sec-butyl chloride solution (4.0 mL, 10.0 mmol, 1.0 equiv, 2.5 M) over 60 min with vigorous stirring. About 1 h after the addition the purple/black heterogeneous mixture was then withdrawn with a syringe and the remaining solids were washed with pentane (2×2.0 mL). The combined pentane solution was filtered through a Teflon syringe filter (PALL PN#4927, 25 mm, 0.2 μm). The resulting solution was titrated following the modified Gilman's method described above to provide 12.89 mL of a 0.69 M solution for an 89% yield.

50 mmol scale: In the glovebox, a 250 mL Schlenk flask was equipped with a glass-coated stir bar, lithium (1.388 g, 200.0 mmol, 4.0 equiv), sodium (13.9 mg, 0.60 mmol, 1 wt %) and capped with a septum. The flask was removed from the glovebox and freshly condensed NH₃ (40 mL, 5 M) was added at −78° C. via cannula. After 5 min, the lithium bronze solution was warmed to rt over 45 min where ammonia in an amount of about 8 mL boiled off slowly. The vessel was placed under vacuum (0.05 torr) to remove the remainder of ammonia over 60 min providing lithium dendrites. The flask was backfilled with argon and the septum was removed briefly for about 30 s followed by carefully scraping the lithium dendrites off the walls to the bottom of the vessel with a metal spatula. The flask was lowered into a preheated oil bath at 37° C. Pentane was added (30.0 mL) followed by the dropwise addition of a sec-butyl chloride solution (20.0 mL, 50.0 mmol, 1.0 equiv, 2.5 M) over 60 min with vigorous stirring. About 1 h after the addition the purple/black heterogeneous mixture was transferred via cannula to a frit containing celite and was filtered. The remaining solids were washed with pentane (2×10.0 mL) transferred via cannula to the celite pad. The resulting filtrate was titrated following the modified Gilman's method described above to provide 51.82 mL of a 0.87 M solution for a 91% yield.

### General Procedure A for the Preparation of Organolithium Reagents

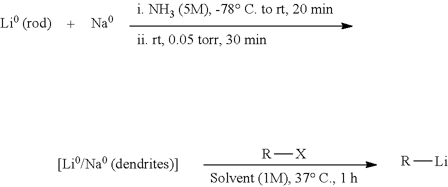

In the glovebox, a 10 mL Schlenk flask was equipped with a glass-coated stir bar, lithium (69.4 mg, 10.0 mmol, 4.0 equiv), sodium (0.7 mg, 0.03 mmol, 1 wt %) and capped with a septum. The flask was removed from the glovebox and freshly condensed NH₃ (2.0 mL, 5 M) was added at −78° C. via cannula. After about 5 min, the lithium bronze solution was warmed to rt over 20 min where ammonia in an amount of about 0.5 mL boiled off slowly. The vessel was placed under vacuum (0.05 ton) to remove the remainder of ammonia over 30 min providing lithium dendrites. The flask was backfilled with argon and the septum was removed briefly for about 10 s followed by carefully scraping the lithium dendrites off the walls to the bottom of the vessel with a metal spatula. The flask was lowered into a preheated oil bath at 37° C. Solvent was added (1.5 mL, 1 M) followed by the dropwise addition of an organohalide solution (1.0 mL, 2.5 mmol, 1.0 equiv, 2.5 M) over 60 min with vigorous stirring. After a minimum of about 1 h after the addition, the purple/black heterogeneous mixture was then withdrawn with a syringe and the remaining solids were washed with solvent (2×1.0 mL). The combined solvent solution was filtered through a Teflon syringe filter (Restek Cat#26142-248, 13 mm, 0.22 μm). The resulting solution was titrated by modifying a known procedure by Gilman.

A scintillon vial was charged with DI water (10 mL) and a magnetic stir bar followed by sparging with argon for 10 min. The vial was fitted with a septum and an aliquot of the organolithium reagent (0.50 mL) was added in one portion. The solution was titrated with standard acid (HCl, 0.242 M) using phenolphthalein as indicator (2-3 drops) to give the total base. A second 5 mL flask was equipped with a magnetic stir bar, capped with a septum, and purged. The flask was charged with diethyl ether (3 mL) and 1,2-dibromoethane (200 μL). An aliquot of organolithium reagent (0.50 mL) was added dropwise with vigorous stifling. After about 5 min, DI water (2 mL) was added, and the solution was titrated with standard acid (HCl, 0.242 M) using phenolphthalein as indicator (2-3 drops) to give the residual base. The active base of the organolithium reagent was then calculated by subtracting the residual base from the total base. The yield of the organolithium reagent was determined using the volume of the solution and concentration of active base. The volume of the solution was calculated by measuring the mass and density prior to titration.

### General Procedure B for the Preparation of Organolithium Reagents

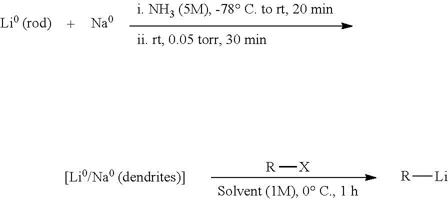

In the glovebox, a 10 mL Schlenk flask was equipped with a glass-coated stir bar, lithium (69.4 mg, 10.0 mmol, 4.0 equiv), sodium (0.7 mg, 0.03 mmol, 1 wt %) and capped with a septum. The flask was removed from the glovebox and freshly condensed NH₃ (2.0 mL, 5 M) was added at −78° C. via cannula. After 5 min, the lithium bronze solution was warmed to rt over 20 min where ammonia in an amount of about 0.5 mL boiled off slowly. The vessel was placed under vacuum (0.05 torr) to remove the remainder of ammonia over 30 min providing lithium dendrites. The flask was backfilled with argon and the septum was removed briefly for about 10 s followed by carefully scraping the lithium dendrites off the walls to the bottom of the vessel with a metal spatula. The flask was lowered into an ice bath at 0° C. Solvent was added (1.5 mL, 1 M) followed by the dropwise addition of an organohalide solution (1.0 mL, 2.5 mmol, 1.0 equiv, 2.5 M) over 15 min with vigorous stirring. After a minimum of about 30 min after the addition, the purple/black heterogeneous mixture was then withdrawn with a syringe and the remaining solids were washed with solvent (2×1.0 mL). The combined solvent solution was filtered through a Teflon syringe filter (Restek Cat#26142-248, 13 mm, 0.22 μm). The resulting solution was titrated as described above.

One skilled in the art will appreciate further features and advantages of the disclosures based on the provided for descriptions and embodiments. Accordingly, the inventions are not to be limited by what has been particularly shown and described. All publications and references cited herein are expressly incorporated herein by reference in their entirety.

Some non-limiting claims are provided below.

What is claimed is:

1. A method of forming an alkali dendrite, comprising:
combining an alkali metal with a solvent to form an alloy; and
removing the solvent from the alloy to form a alkali dendrite.

2. The method of claim 1, wherein combining the alkali metal with the solvent comprises dissolving the alkali metal in the solvent.

3. The method of claim 1, wherein the alkali is lithium.

4. The method of claim 3, wherein the lithium is a lithium rod. The method of claim 1, wherein the alkali is one or more of sodium or potassium.

6. The method of claim 1, wherein the solvent comprises liquid ammonia.

7. The method of claim 1, wherein the solvent comprises one or more of Hexmethylphosphoramide (HMPA) and different degrees of amines.

8. The method of claim 1, further comprising keeping a temperature of the alloy substantially constant during the combining step.

9. The method of claim 8, further comprising stirring the alloy to evaporate an amount of the solvent from the alloy. The method of claim 9, wherein an amount of the solvent removed from the alloy can be in approximately a range of about 99% to about 100% with respect to a total amount of the solvent in the alloy.

11. The method of claim 1, wherein the solvent is removed by using a vacuum.

12. The method of claim 1, wherein a surface area of the alkali dendrite is about 100 times greater than a surface area of a conventional alkali powder.

13. The method of claim 1, wherein a surface area of the alkali dendrite is about 2,950 times greater than a surface area of the alkali metal.

14. The method of claim 1, wherein a bulk morphology of the alkali dendrite is agglomerated in a dendritic form. The method of claim 1, wherein a reactivity of the alkali dendrite is about 19 times greater than a reactivity of a conventional alkali powder.

16. The method of claim 1, wherein a reactivity of the alkali dendrite is about 199 times greater than a reactivity of the alkali metal.

17. The method of claim 1, further comprising reacting the alkali dendrite with an organic halide to form an organometallic reagent.

18. The method of claim 17, further comprising synthetically transforming the organometallic reagent by alkylation to generate a yield of about 90% or more

19. An organo-alkali metal reagent synthesized using the alkali dendrite of claim 1. A synthetically transformed compound formed from the organometallic reagent of claim 17.
